(19) United States
(12) Patent Application Publication
(10) Pub. No.: US 2025/0001135 A1
(43) Pub. Date: Jan. 2, 2025

(54) Catheter Securement Device

(71) Applicant: Becton, Dickinson and Company, Franklin Lakes, NJ (US)

(72) Inventors: Todd L. Schwendiman, South Jordan, UT (US); Jonathan Rutledge, Layton, UT (US); Michael Issac Valencia, Tooele, UT (US); Richard John Fox, Branchburg, NJ (US); Aniruddha Chatterjee, Randolph, NJ (US)

(73) Assignee: Becton, Dickinson and Company, Franklin Lakes, NJ (US)

(21) Appl. No.: 18/709,333

(22) PCT Filed: Nov. 18, 2022
(86) PCT No.: PCT/US2022/050372

§ 371 Date: May 10, 2024

Related U.S. Application Data
(60) Provisional application No. 63/281,809, filed on Nov. 22, 2021.

(51) Int. Cl. A61M 25/02

(52) U.S. Cl. CPC A61M 25/02; A61M 2025/0253

(57) ABSTRACT

A catheter securement device comprising a base (110) in contact with the skin of a patient and a pad for securing a catheter, a catheter assembly or a portion of a catheter assembly. The base includes perforations (120) for case of removal of the catheter securement device from the skin of a patient without disrupting the catheter insertion region.

### TECHNICAL FIELD

The present disclosure relates to intravenous catheters. More specifically, this disclosure relates to systems and methods of using a sterile intravenous catheter securement device or dressing to permit sterile securement of the intravenous catheter to a patient.

### BACKGROUND

Catheters are commonly used for a variety of infusion therapies. For example, catheters are used for infusing fluids, such as normal saline solution, various medicaments, and total parenteral nutrition into a patient, withdrawing blood from a patient, as well as monitoring various parameters of the patient's vascular system.

Catheters are commonly introduced into the vasculature of a patient as part of an intravenous catheter assembly. The catheter assembly generally includes a catheter hub, which supports the catheter, the catheter hub being coupled to a needle hub which supports an introducer needle. The introducer needle is extended and positioned within the catheter such that a beveled portion of the needle is exposed beyond a tip of the catheter. The beveled portion of the needle is used to pierce the skin of the patient to provide an opening whereby to insert the needle in the vasculature of the patient. Following insertion and placement of the catheter, the introducer needle is removed from the catheter thereby providing intravenous access to the patient.

Catheter-related bloodstream infections are caused by the colonization of microorganisms in patients with intravascular catheters and I.V. access devices. These infections are an important cause of illness and excess medical costs, as approximately 250,000 catheter-related bloodstream infections occur in United States intensive care units each year. In addition to the monetary costs, these infections are associated with anywhere from 20,000 to 100,000 deaths each year.

Despite guidelines to help reduce healthcare associated infections (HAIs), catheter-related bloodstream infections continue to plague our healthcare system. The 10 most common pathogens (accounting for 84% of any HAIs) were coagulase-negative staphylococci (15%), Staphylococcus aureus (15%), Enterococcus species 12%), Candida species (11%), Escherichia coli (10%), Pseudomonas aeruginosa (8%), Klebsiella pneumoniae (6%), Enterobacter species (5%), Acinetobacter baumannii (3%), and Klebsiella oxytoca (2%). The pooled mean proportion of pathogenic isolates resistant to antimicrobial agents varied significantly across types of HAI for some pathogen-antimicrobial combinations. As many as 16% of all HAIs were associated with the following multidrug-resistant pathogens: methicillin-resistant S. aureus (8% of HAIs), vancomycin-resistant Enterococcus faecium (4%), carbapenem-resistant P. aeruginosa (2%), extended-spectrum cephalosporin-resistant K. pneumoniae (1%), extended-spectrumcephalosporin-resistant E. coli (0.5%), and carbanpenem-resistant A. baumannii, K. pneumoniae, K. oxytoca, and E. coli (0.5%) antimicrobial-resistant pathogens.

Peripheral Intravenous catheters (PIVC) are placed into a peripheral vein for venous access to administer intravenous therapy such as medication fluids. PIVC catheters are often used when undergoing any recovery or medical procedure requiring administration of medication fluids at a controlled rate. The PIVC catheter is commonly secured to the skin of a patient with a stabilization dressing, which is commonly adhered to either the skin of the patient, the catheter itself or both.

During removal and replacement of the stabilization dressing, the adhered dressing is often difficult to replace without moving the catheter itself, which is still attached to the skin or the catheter. Excessive movement or destabilization of the catheter can lead to rupture or irritation of the skin at the insertion site, which can result in phlebitis and other similar complications. It is recommended to remove and replace stabilization dressings every 72 to 96 hours by the US Center for Disease Control to reduce the risk of phlebitis ad bloodstream infection.

Thus, there is a need in the art to provide a stabilization dressing having ease of removal from the skin of a patient or the catheter without disrupting the insertion site.

### SUMMARY

A first aspect of the present disclosure relates to a catheter securement device having a base including a distal end, a proximal end, a first end and a second end. The first end and second end define a width Wb, the first end and second end perpendicular to the distal end, and a perforation is positioned between the first end and second end and perpendicular to the width Wb, the base having a skin-contacting bottom surface and a top surface opposite the bottom surface. A first pad is disposed over the top surface of the base, the first pad having a medial pad edge adjacent to and over the perforation of the base. A second pad is disposed over the top surface of the base, the second pad having a medial pad edge adjacent to and over the perforation of the base, the medial pad edge of the first pad abutting the medial pad edge of the first pad. The first pad and second pad forming a disunited body. The disunited body is configured to separate upon application of a shear force on the base such that the first pad is separated from the second pad. The first pad and second pad are non-removably adhered to the top surface of the base.

In some embodiments, wherein the base has a rectangular, square, oval or circular shape.

In some embodiments, the perforation extends from the proximal end to the distal end and is configured to separate the base upon application of a lateral shear force to either or both of the first end and second end. In some embodiments, the perforation is positioned at half of the width Wb.

In some embodiments, the skin-contacting bottom surface includes a medical-grade adhesive. In other embodiments, the skin-contacting bottom surface includes a medical-grade adhesive and an antiseptic or antimicrobial agent.

In some embodiments, the medical-grade adhesive has an adhesive strength which secures to the skin of a patient and can also be removed by application of a shear force laterally to one or both of the first end and second end of the base.

In some embodiments, the proximal end of the base includes a first extension extending proximally from the proximal end and a second extension extending proximally from the proximal end, the first extension and second extension configured as pull tabs.

In some embodiments, each of the first pad and second pad have common a distal end and a common proximal end, from the common proximal end extends a cavity having a proximal portion and a distal portion. In some embodiments, the proximal portion of the cavity has a width Wp greater than a width Wd of the distal portion.

In some embodiments, the proximal portion and distal portion of the cavity is configured to receive a catheter extension set. In some embodiments, the proximal portion and distal portion of the cavity is configured to receive at least a distal tip of a catheter.

In some embodiments, the catheter extension set is removably secured to the base or disunited body by medical-grade tape.

In some embodiments, the common proximal end of the first pad and second pad are at an angle.

In some embodiments, the application of a lateral force to the first extension severs the perforation.

In some embodiments, the first pad and second pad have a thickness greater than a thickness of the base.

A second aspect of the present disclosure relates to a method of use of the catheter securement device of the present disclosure having the steps of: gripping a first extension and second extension of the base; and, applying a lateral shear force to the base such that the perforation tears thereby severing the base at the perforation. In some embodiments, the method further includes an catheter extension set or catheter device system disposed over the base is not disrupted or lifted by application of the lateral shear force and tearing of the perforation.

### BRIEF DESCRIPTION OF THE DRAWINGS

FIG. 1A illustrates a perspective of a catheter device system vascular common in the art;

FIG. 1B illustrates a side view of a catheter extension set common in the art;

FIG. 2 illustrates a top view of a catheter extension set disposed over a catheter securement device in accordance with one or more embodiments of the present disclosure;

FIG. 3 illustrates a top view of a catheter extension set disposed over a catheter securement device in accordance with one or more embodiments of the present disclosure;

FIG. 4 illustrates a perspective view of a catheter securement device in accordance with one or more embodiments of the present disclosure;

FIG. 5 illustrates a perspective view of a catheter securement device in accordance with one or more embodiments of the present disclosure;

FIG. 6 illustrates a perspective view of a torn catheter securement device in accordance with one or more embodiments of the present disclosure; and

FIGS. 7A-7D illustrates a perspective view of an antiseptic or antimicrobial agent applied to various surfaces of a catheter securement device in accordance with one or more embodiments of the present disclosure. FIG. 7A illustrates a perspective view of an antiseptic or antimicrobial agent applied to the top of a comfort pad foam adhesive layer. FIG. 7B illustrates a perspective view of an antiseptic or antimicrobial agent applied to the inside of a luer lock-nut well. FIG. 7C illustrates a perspective view of an antiseptic or antimicrobial agent applied within the structural foam of the device. FIG. 7D illustrates a perspective view of an antiseptic or antimicrobial agent applied underneath an adhesive layer in contact with the skin of patient.

### DETAILED DESCRIPTION

Before describing several exemplary embodiments of the disclosure, it is to be understood that the disclosure is not limited to the details of construction or process steps set forth in the following description. The disclosure is capable of other embodiments and of being practiced or being carried out in various ways.

For purposes of the description hereinafter, the terms “proximal”, “distal”, “longitudinal”, and derivatives thereof shall relate to the disclosure as it is oriented in the drawing figures. However, it is to be understood that the disclosure may assume alternative variations and step sequences, except where expressly specified to the contrary. It is also to be understood that the specific devices and processes illustrated in the attached drawings, and described in the following specification, are simply exemplary embodiments of the disclosure. Hence, specific dimensions and other physical characteristics related to the embodiments disclosed herein are not to be considered as limiting.

As used herein, the use of “a,” “an,” and “the” includes the singular and plural.

As used herein, the use of “catheter securement device” includes dressings for catheter securement.

As used herein, the term “medical device” refers to common medical devices having threaded or interlocking connections, the connections having corresponding mating elements. By way of example but not limitation, a syringe may have a threaded connection which releasably interlocks with a secondary medical device such as a needless connector of a catheter, an IV line and the like. The threaded connection may include a lumen defining a fluid path surrounded by a protruding wall having the threaded means for attaching to the secondary medical device.

As would be readily appreciated by skilled artisans in the relevant art, while descriptive terms such as “thread”, “taper”, “tab”, “wall”, “proximal”, “side”, “distal” and others are used throughout this specification to facilitate understanding, it is not intended to limit any components that can be used in combinations or individually to implement various aspects of the embodiments of the present disclosure.

Embodiments of the present disclosure are directed to a catheter securement device comprising a base in contact with the skin of a patient and a pad for securing a catheter, a catheter assembly or a portion of a catheter assembly. The base includes perforations for ease of removal of the catheter securement device from the skin of a patient without disrupting the catheter insertion region.

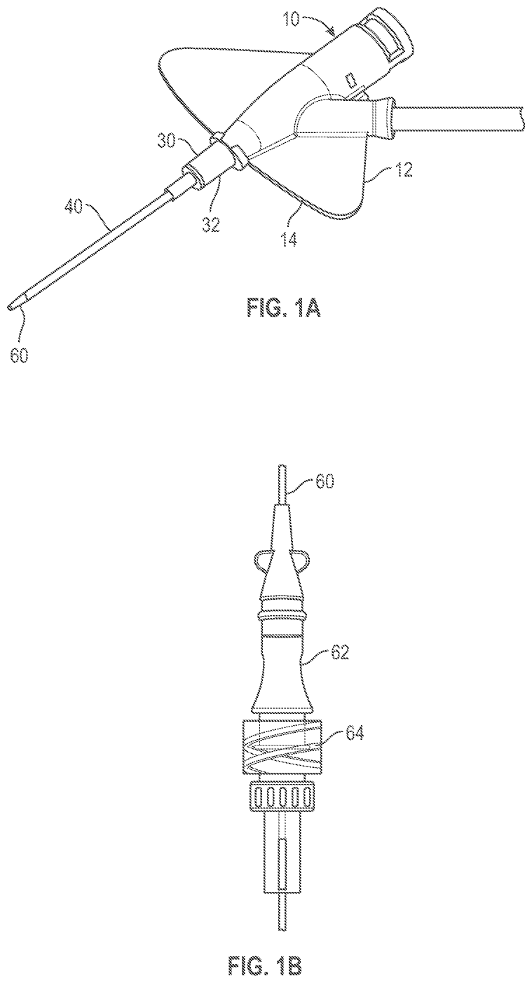

In general, as shown in FIG. 1A, a catheter device system 10 in accordance with the prior art provides access to the vasculature of a patient. In some embodiments, catheter device system comprises 10 a catheter hub 30 which supports a catheter tube 40. The catheter tube 40 extends outwardly from catheter hub 30 and is in fluid communication with the catheter hub 40.

In some embodiments, catheter device system 10 further comprises a needle hub which supports an introducer needle 60. The introducer needle 60 is threadedly positioned through catheter hub 30 and catheter tube 40 such that a beveled tip of needle extends beyond catheter tip. The beveled tip provides a cutting surface to penetrate the patient's skin and provide access to the patient's vasculature. In some embodiments, once the catheter 40 is fully inserted into vasculature, the introducer needle 60 and needle hub is removed providing intravenous access to the patient via catheter 40 and catheter hub adapter 30. The inserted catheter 40 is at least partially within the transdermal surface or intravascular surface of the skin of the patient. In some embodiments, the catheter device system 10 further includes wing elements 12 or extensions which protrude outwardly from the catheter hub 30. In some embodiments, a distal edge 14 of the wing elements 12 are angled or perpendicular to the catheter hub 30. In some embodiments, a distal tip 32 of the catheter hub 30 has a substantially cylindrical shape having a diameter and a length.

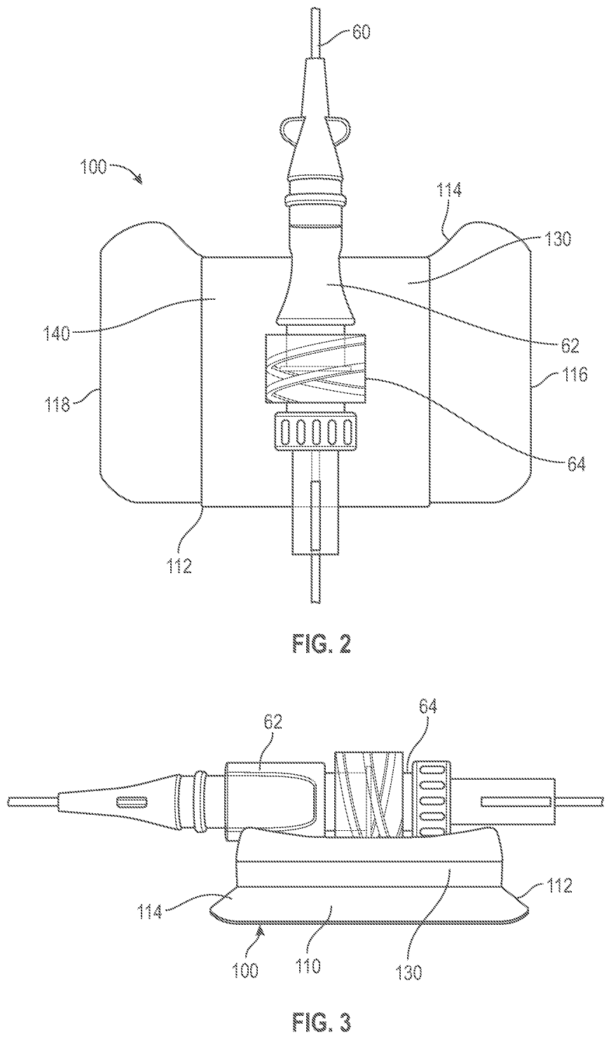

In general, as shown in FIGS. 1B and 2, a catheter extension set 50 replaces the catheter device system 10 after insertion and subsequent removal of the catheter device system 10 upon insertion into the skin of a patient. In some embodiments, the catheter extension set 50 comprises the introducer needle 60 and a hub 62 connected to a needleless connector 64. In some embodiments, the hub 62 and needleless connector 64 have a substantially cylindrical shape having a diameter and a length.

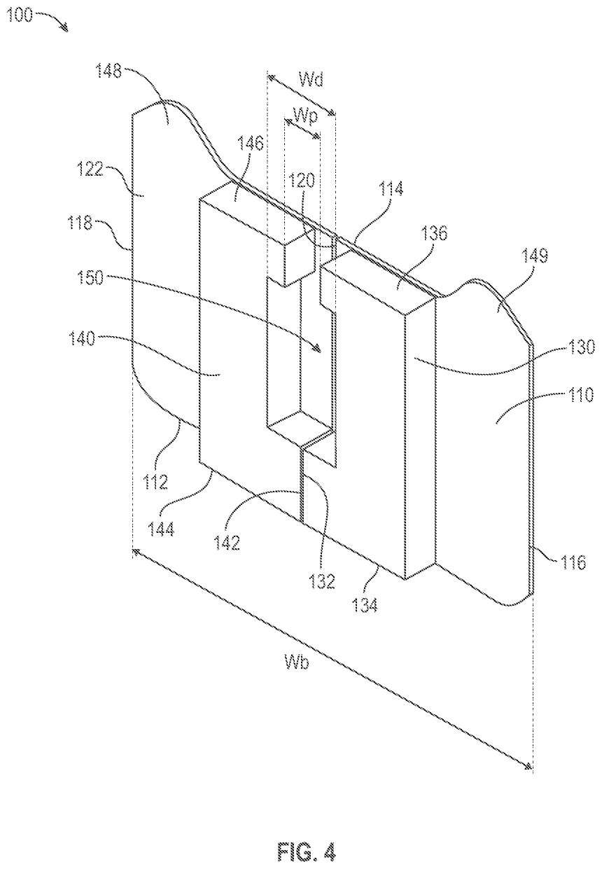

FIGS. 3 through 6 illustrate embodiments of a catheter securement device 100 comprising a base 110 having a rectangular shape. In some embodiments, the base 110 has a square, oval or more generally a circular shape. The base 110 comprises a distal end 112, a proximal end 114, a first end 116 and a second end 118. The first end 116 and second end 118 define a width Wb, and the first end 116 and second end 118 are perpendicular to the distal end 112. Stated differently, in embodiments where the base 110 has a rectangular or square shape, the distal end 112 and proximal end 114 are opposite faces of the rectangular or square shape, and the first end 116 and second end 118 are adjacent to the distal end 112 and proximal end 114. In embodiments where the base 110 has an oval or circular shape, the distal end 112 and proximal end 114 are opposite edges of the oval or circular shape, and the first end 116 and second end 118 are perpendicular to the distal end 112 and proximal end 114

In some embodiments, a perforation 120 is positioned between the first end 116 and second end 118 and is perpendicular to the width Wb. The perforation 120 extends from the proximal end 114 to the distal end 112 and is configured to separate the base 110 upon application of a lateral shear force to either or both of the first end 116 and second end 118. In some embodiments, the perforation 120 is positioned at half of the width Wb.

The base 110 has a skin-contacting bottom surface (not shown) and a top surface 122 opposite the bottom surface. The skin-contacting bottom surface in some embodiments comprises a medical-grade adhesive. In some embodiments, the bottom surface comprises a medical-grade adhesive and an antiseptic or antimicrobial agent. The medical-grade adhesive has an adhesive strength which secures to the skin of a patient and can also be removed by application of a shear force laterally to one or both of the first end 116 and second end 118 of the base 110. Thus, the medical-grade adhesive and bottom surface of the base 110 is removably attached to the skin of a patient and can be removed by application of a shear force to one or both of the first end 116 and second end 118 of the base 110.

A first pad 130 and a second pad 140 are disposed over the top surface 122 of the base 110. Each of the first pad 130 and second pad 140 have a thickness which is greater than a thickness of the base 110. Each of the first pad 130 and second pad 140 of some embodiments are made of a foam material. Each of the first pad 130 and second pad 140 are non-removably adhered to the top surface 122 of the base 110.

The first pad 130 has a medial pad edge 132 which is positioned adjacent to and over the perforation 120 of the base 110. Likewise, the second pad 140 has a medial pad edge 142 which is positioned adjacent to and over the perforation of the base 110. The medial pad edge 132 of the first pad abuts the medial pad edge 142 of the second pad 140 forming a disunited (essentially non-integral) body. In some embodiments, the disunited body formed by the first pad 130 and second pad 140 is rectangular, square, oval or otherwise circular shape. The disunited body is configured to separate upon application of a shear force on the base 110 such that the first pad 130 is separated from the second pad 140.

In some embodiments, an antiseptic or antimicrobial agent is applied to one or more surfaces of the first pad 130 and second pad 140, including surfaces defined by the thickness of the first pad 130 and second pad 140.

Each of the first pad 130 and second pad 140 have a common distal end (134, 144) and a common proximal end (136, 146). From the proximal end (136, 146) of the first pad 130 and second pad 140 extends a cavity 150 having a proximal portion 152 and a distal portion 154, the proximal portion 152 having a width Wp greater than a width Wd of the distal portion 154. The proximal portion 152 and distal portion 154 extending partially the length of the disunited body. The cavity 150 extends the entire thickness of the disunited body.

In embodiments where a catheter extension set 50 is inserted into the skin of a patient, the cavity 150 is configured to receive the catheter extension set 50. By way of example but not limitation, as shown in FIG. 2, the hub 62 of the catheter extension set is seated within the proximal portion 152 of the cavity and the needleless connector 64 is seated within the distal portion 154 of the cavity. The catheter extension set 50 in some embodiments is removably secured to the base 110 or disunited body by medical-grade tape.

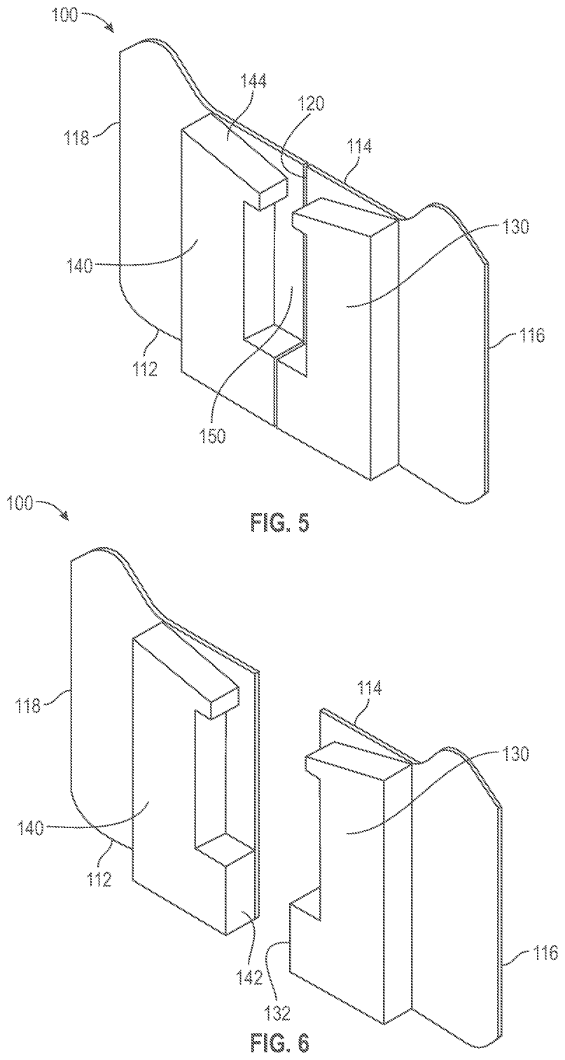

In embodiments where the catheter device system 10 is inserted into the skin of a patient, the cavity 150 is configured to receive at least the distal tip 32 of the catheter device system 10. Furthermore, as best shown in FIG. 5, the proximal end (136, 146) of the first pad 130 and second pad 140 are at an angle to fit the angled wing elements 12 of the catheter device system 10.

In some embodiments, the proximal end 114 of the base 110 further comprises a first extension 148 extending proximally from the proximal end 114 of the base 110 and a second extension 149 extending proximally from the proximal end 114 of the base 110. In some embodiments, the first extension 148 and second extension 149 are configured as pull tabs to facilitate grip points for a user or practitioner to apply the shear force laterally from the base 110. In some embodiments, the bottom surface of the first extension 148 and second extension 149 do not comprise medical-grade adhesive such that the first extension 148 and second extension 149 can be slightly lifted to be gripped better by the user or practitioner during application of the shear force in a lateral direction relative to the base 110. FIG. 6 illustrates a perspective view of a torn catheter securement device in accordance with one or more embodiments of the present disclosure.

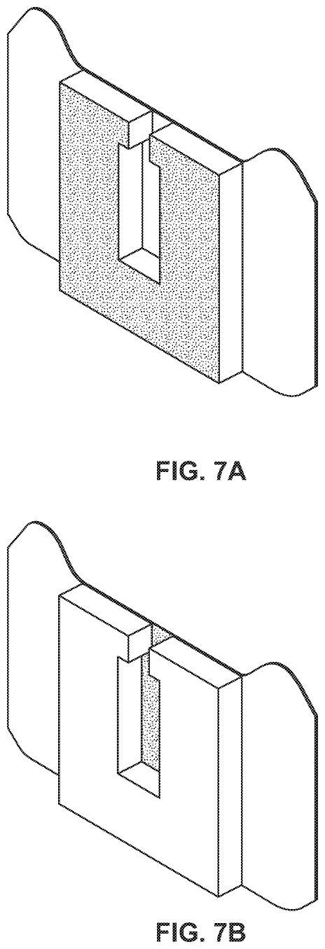

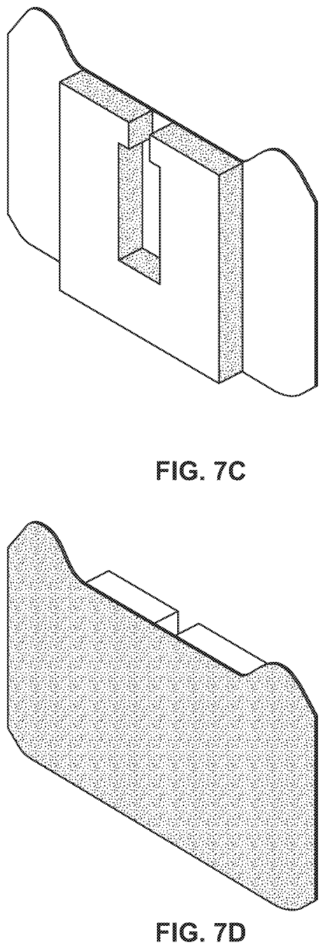

During placement of catheter securement or stabilization device or dressing, the catheter securement or stabilization device or dressing only aids to stabilize the catheter/extension set configuration, while having direct and indirect contact to the skin nearing the insertion site. This leads to possible pooling of bacteria in the devices under the stabilization dressing. To reduce bacteria infection, in one or more embodiments, an antiseptic or antimicrobial agent is incorporated into the adhesive or skin-contacting bottom surface or the top surface of the catheter securement device as shown in FIG. 7. In one or more embodiments, the antiseptic or antimicrobial agent is Chlorhexidine Gluconate (CHG). FIGS. 7A-7D illustrates a perspective view of an antiseptic or antimicrobial agent applied to various surfaces of a catheter securement device in accordance with one or more embodiments of the present disclosure. In one or more specific embodiments, antiseptic or antimicrobial agent, such as CHG, are applied to surfaces including, but not limited to, the top surface of catheter securement device, bottom surface of catheter securement device, and a surface of a luer locknut well, as shown in FIGS. 7A-7D. FIG. 7A illustrates a perspective view of an antiseptic or antimicrobial agent applied to the top of a comfort pad foam adhesive layer. FIG. 7B illustrates a perspective view of an antiseptic or antimicrobial agent applied to the inside of a luer lock-nut well. FIG. 7C illustrates a perspective view of an antiseptic or antimicrobial agent applied within the structural foam of the device. FIG. 7D illustrates a perspective view of an antiseptic or antimicrobial agent applied underneath an adhesive layer in contact with the skin of patient.

The application of an antiseptic or antimicrobial agent, such as CHG, will allow for reduction in risk of insertion site area microbe exposure, which is found to decrease the possibility of contracting phlebitis/CRBSI. A benefit to the antiseptic or antimicrobial agent in the adhesive is it provides protection to surfaces with direct contact to active catheter insertion area skin and peripheral contact of catheters and extension sets in contact. The application of an antiseptic or antimicrobial agent, such as CHG, will allow for the advantageous risk reduction of microbial exposure near insertion site area, which is found to decrease complications such as CRBSI/phlebitis. The antiseptic or antimicrobial agent, such as CHG, antimicrobial properties will help areas with direct and peripheral contact to skin. Implementation of an adhesive incorporating antiseptic or antimicrobial agent, such as CHG, to the surfaces of the device to aid in actively preventing any form of phlebitis/CRBSI through direct and indirect contact. In some embodiments, the antiseptic or antimicrobial agent is incorporated into the medical-grade adhesive.

A method of use of the catheter securement device 100 comprises the steps of: inserting the catheter device system 10 or catheter extension set 50 into the skin of a patient, positioning the base 110 under the catheter device system 10 or catheter extension set 50 such that the catheter device system 10 or catheter extension set 50 is seated within the cavity 150, securing the catheter device system 10 or catheter extension set 50 to the base 110. Upon removal of the catheter securement device 100, the method further comprises the steps of gripping the first extension 148 and second extension 149 of the base 110 and applying a lateral shear force to the base 110 such that the perforation 120 tears and thereby severing the base 110 at the perforation 120. Because the catheter securement device 100 can be removed by merely applying a lateral shear force, the catheter device system 10 or catheter extension set 50 is not disrupted or lifted and the insertion site is also not disrupted. A new catheter securement device 100 can be placed under the catheter device system 10 or catheter extension set 50.

While the present disclosure has been shown and described with reference to certain exemplary embodiments thereof, it will be understood by those skilled in the art that various changes in form and details may be made therein without departing from the spirit and scope of the embodiments of the present disclosure. Also, the inner and/or the outer housing of the disinfection cap can be single shot molded, or made by other suitable process. Furthermore, any of the features or elements of any exemplary implementations of the embodiments of the present disclosure as described above and illustrated in the drawing figures can be implemented individually or in any combination(s) as would be readily appreciated by skilled artisans without departing from the spirit and scope of the embodiments of the present disclosure.

In addition, the included drawing figures further describe non-limiting examples of implementations of certain exemplary embodiments of the present disclosure and aid in the description of technology associated therewith. Any specific or relative dimensions or measurements provided in the drawings other as noted above are exemplary and not intended to limit the scope or content of the inventive design or methodology as understood by artisans skilled in the relevant field of invention.

Reference throughout this specification to “one embodiment,” “certain embodiments,” “one or more embodiments” or “an embodiment” means that a particular feature, structure, material, or characteristic described in connection with the embodiment is included in at least one embodiment of the disclosure. Thus, the appearances of the phrases such as “in one or more embodiments,” “in certain embodiments,” “in one embodiment” or “in an embodiment” in various places throughout this specification are not necessarily referring to the same embodiment of the disclosure. Furthermore, the particular features, structures, materials, or characteristics may be combined in any suitable manner in one or more embodiments.

Although the disclosure herein has provided a description with reference to particular embodiments, it is to be understood that these embodiments are merely illustrative of the principles and applications of the present disclosure. It will be apparent to those skilled in the art that various modifications and variations can be made to the method and apparatus of the present disclosure without departing from the spirit and scope of the disclosure. Thus, it is intended that the present disclosure include modifications and variations that are within the scope of the appended claims and their equivalents.

What is claimed is:

1. A catheter securement device, comprising:
a base having a distal end, a proximal end, a first end and a second end, the first end and second end defining a width Wb, the first end and second end perpendicular to the distal end, and a perforation positioned between the first end and second end and perpendicular to the width Wb, the base having a skin-contacting bottom surface and a top surface opposite the bottom surface; and,
a first pad disposed over the top surface of the base, the first pad having a medial pad edge adjacent to and over the perforation of the base; and,
a second pad disposed over the top surface of the base, the second pad having a medial pad edge adjacent to and over the perforation of the base, the medial pad edge of the first pad abutting the medial pad edge of the first pad; the first pad and second pad forming a disunited body, the disunited body configured to separate upon application of a shear force on the base such that the first pad is separated from the second pad;
wherein the first pad and second pad are non-removably adhered to the top surface of the base.

2. The catheter securement device of claim 1, wherein the base has a rectangular, square, oval or circular shape.

3. The catheter securement device of claim 1, wherein the perforation extends from the proximal end to the distal end and is configured to separate the base upon application of a lateral shear force to either or both of the first end and second end.

4. The catheter securement device of claim 1, wherein the perforation is positioned at half of the width Wb.

5. The catheter securement device of claim 1, wherein the skin-contacting bottom surface comprises a medical-grade adhesive.

6. The catheter securement device of claim 5, wherein the skin-contacting bottom surface comprises a medical-grade adhesive and an antiseptic or antimicrobial agent.

7. The catheter securement device of claim 5, wherein the medical-grade adhesive has an adhesive strength which secures to the skin of a patient and can also be removed by application of a shear force laterally to one or both of the first end and second end of the base.

8. The catheter securement device of claim 6, wherein the antiseptic or antimicrobial agent is incorporated into the medical-grade adhesive.

9. The catheter securement device of claim 6, wherein the antiseptic or antimicrobial agent is chlorhexidine gluconate.

10. The catheter securement device of claim 1, wherein the proximal end of the base comprises a first extension extending proximally from the proximal end and a second extension extending proximally from the proximal end, the first extension and second extension configured as pull tabs.

11. The catheter securement device of claim 1, wherein each of the first pad and second pad have common a distal end and a common proximal end, from the common proximal end extends a cavity having a proximal portion and a distal portion.

12. The catheter securement device of claim 11, wherein the proximal portion of the cavity has a width Wp greater than a width Wd of the distal portion.

13. The catheter securement device of claim 11, wherein the proximal portion and distal portion of the cavity is configured to receive a catheter extension set.

14. The catheter securement device of claim 13, wherein the catheter extension set is removably secured to the base or disunited body by medical-grade tape.

15. The catheter securement device of claim 11, wherein the proximal portion and distal portion of the cavity is configured to receive at least a distal tip of a catheter.

16. The catheter securement device of claim 1, wherein the common proximal end of the first pad and second pad are at an angle.

17. The catheter securement device of claim 8, wherein application of a lateral force to the first extension severs the perforation.

18. The catheter securement device of claim 1, wherein the first pad and second pad have a thickness greater than a thickness of the base.

19. A method of use of the catheter securement device of claim 1 comprising the steps of:
gripping a first extension and second extension of the base; and,
applying a lateral shear force to the base such that the perforation tears thereby severing the base at the perforation.

20. A method of claim 19, wherein a catheter extension set or catheter device system disposed over the base is not disrupted or lifted by application of the lateral shear force and tearing of the perforation.
